(19) United States
(12) Patent Application Publication
(10) Pub. No.: US 2026/0248817 A1
(43) Pub. Date: Aug. 27, 2026

(54) COMPOSITIONS COMPRISING CETYLATED MYRISTIC ACID, OLEIC ACID, OR A MIXTURE THEREOF, AND VITAMIN D, AND MEDICAL USES THEREOF

(71) Applicant: PHARMANUTRA S.P.A., PISA (IT)

(72) Inventors: Andrea LACORTE, PISA (IT); Germano TARANTINO, PISA (IT); Elisa BRILLI, PISA (IT)

(21) Appl. No.: 19/162,302

(22) PCT Filed: Mar. 7, 2024
(86) PCT No.: PCT/IB2024/052210

§ 371 Date: Sep. 4, 2025

(30) Foreign Application Priority Data
Mar. 7, 2023 (IT) 102023000004227

(51) Int. Cl. A61K 31/593; A61K 9/00; A61K 31/231; A61K 47/44; A61P 19/00

(52) U.S. Cl. CPC A61K 31/593; A61K 9/0053; A61K 31/231; A61K 47/44; A61P 19/00

(57) ABSTRACT

It is an object of the present invention a composition comprising a mixture comprising or, alternatively, consisting of at least one cetylated fatty acid or a mixture of cetylated fatty acids, a vitamin D and, optionally, an antioxidant. Further, it is an object of the present invention a composition comprising a mixture comprising or, alternatively, consisting of at least one cetylated fatty acid or a mixture of cetylated fatty acids, a vitamin D and, optionally, an antioxidant, said composition being for use in a method for bone proliferation-regeneration, particularly in a method of promoting and improving bone proliferation-regeneration; or said composition being for use in a method of treatment, preferably preventive, of osteoporosis, preferably curative of bone fracture; for use in a method of treatment in prosthetic surgery, bone growth, osteoarthritis, consolidation post-fracture, joint pain, musculoskeletal pain, including, but not limited to, those of an inflammatory nature. Additionally, it is an object of the present invention a composition comprising a mixture comprising, or, alternatively, consisting of at least one cetylated fatty acid or a mixture of cetylated fatty acids, a vitamin D, and optionally an antioxidant, said composition being for use also in a method of treatment of gastric mucosal protection and regulation of blood glucose levels. Finally, it is an object of the present invention pharmaceutical or nutraceutical formulations, or formulations for supplements or foods, or cosmetic formulations containing said mixture and/or composition, for example in the form of topical or oral formulations (in short, the formulations of the invention).

It is an object of the present invention a composition comprising a mixture comprising or, alternatively, consisting of at least one cetylated fatty acid or a mixture of cetylated fatty acids, a vitamin D and, optionally, an antioxidant. Further, it is an object of the present invention a composition comprising a mixture comprising or, alternatively, consisting of at least one cetylated fatty acid or a mixture of cetylated fatty acids, a vitamin D and, optionally, an antioxidant, said composition being for use in a method for bone proliferation-regeneration, particularly in a method of promoting and improving bone proliferation-regeneration; or said composition being for use in a method of treatment, preferably preventive, of osteoporosis, preferably curative of bone fracture; for use in a method of treatment in prosthetic surgery, bone growth, osteoarthritis, consolidation post-fracture, joint pain, musculoskeletal pain, including, but not limited to, those of an inflammatory nature. Additionally, it is an object of the present invention a composition comprising a mixture comprising, or, alternatively, consisting of at least one cetylated fatty acid or a mixture of cetylated fatty acids, a vitamin D, and optionally an antioxidant, said composition being for use also in a method of treatment of gastric mucosal protection and regulation of blood glucose levels. Finally, it is an object of the present invention pharmaceutical or nutraceutical formulations, or formulations for supplements or foods, or cosmetic formulations containing said mixture and/or composition, for example in the form of topical or oral formulations (in short, the formulations of the invention).

Compositions comprising mixtures of cetylated fatty acids and their use in the treatment of osteoarthritis and joint inflammation are known in the art. For example, patent documents WO2004/084829 A2 and WO2017/029580 A1 describe compositions comprising cetylated fatty acids and, optionally, mixtures of tocopherols (vitamin E, fat-soluble) as antioxidants in a weight % from 1% to 5%, with respect to the total weights of the compositions.

It is known that osteoarthritis is a chronic joint disease characterized by the progressive loss of articular cartilage and the formation of osteophytes (bone hypertrophy). These progressive changes in the joints can lead over time to the gradual development of symptoms such as pain and joint stiffness with major repercussions on mobility and skeletal support. Osteoarthritis can be classified as primary if without precise cause, or secondary if it occurs instead as a result of events that have disrupted the cartilage microenvironment. Joint pain is the main symptom of the disease and a limiting factor in quality of life.

Joint pain refers to the localized painful sensation within one or more joints of the body. The disorder recognizes very different causes including the degenerative and progressive process affecting cartilage typical of osteoarthritis, as a result of joint or peri-articular injuries, accidents or trauma (e.g., fractures, dislocations, sprains, etc.) or it may also depend on the presence of inflammation (arthritis). Joint pain can also result from infection and a variety of other pathologies including, for example, rheumatoid arthritis, bursitis, tendonitis, and others.

It is known that musculoskeletal pain generally manifests as bone pain, affecting the joints but also tendons and ligaments, or muscle pain. Discomfort at cervical muscle, at muscles of the spine and back, at knees, just to name a few examples. In some cases the pain passes after a short time, in others it even becomes chronic affecting daily life and even mood.

It is known that muscle pain can be due to several causes: injury, trauma, strenuous exercise, strenuous work, or poor posture. Muscle pain is manifested by generally widespread pain involving the muscles and may be due to overstressing or over-exercising. Joint pain, on the other hand, is typically what is felt when loading the joint, in a very localized manner and is felt not only during exercise and movement but also at rest.

However, there remains a high level of interest among industry and market participants for new, ever-improving and useful compositions for administration to all categories of subjects such as, for example, adults, athletes and sportspersons.

The technical problem that the present invention addresses and solves is to provide mixtures and/or compositions comprising cetylated fatty acids that are improved over known compositions and that are, at the same time, capable of promoting or improving bone proliferation. In addition, the mixtures and/or compositions comprising cetylated fatty acids must be effective, particularly in the treatment of both osteoarthritis, and joint and musculoskeletal disorders or pain, bone proliferation, fracture consolidation, and bone regeneration, that are also free of side effects, well tolerated, stable over time with respect to the action of oxidizing agents, and, in addition, cost-effective and easy to produce.

The present invention provides compositions (pharmaceutical compositions, medical device compositions Reg. EU 745/2017, novel food (novel food in accordance with Regulation (EU) 2015/2283 amending Implementing Regulation (EU) 2017/2470), dietary supplements or cosmetic compositions, in short: compositions of the invention, comprising at least one cetylated fatty acid or a mixture of cetylated fatty acids, one vitamin D and, optionally, at least one antioxidant.

In fact, the presence of vitamin D is capable of increasing the effectiveness of the compositions of the invention in the treatment of diseases, symptoms or disorders listed from (i) to (vi) below in this description.

Specifically, as shown in FIGS. 1-3, the mixture and/or composition of the invention is capable of significantly increasing the mineralization levels of human mesenchymal stem cells (hMSCs) through the combination of at least one cetylated fatty acid, or a mixture of cetylated fatty acids, and vitamin D, preferably vitamin D3.

Preferably, the presence of the antioxidant in the mixtures and/or compositions helps to make them stable over time, in that the titer of the active ingredient (and in particular of said at least one cetylated fatty acid) remains virtually unchanged over time, preserving the cetylated fatty acids from the action of oxidizing agents. Consequently, the presence of the antioxidant can make the mixtures and/or compositions of the invention improved with respect to their efficacy, transportation, storage, and time and mode of preservation from the time of first opening the package containing the mixture or composition of the invention.

In addition, the presence of the antioxidant in a lower weight % than in cetylated fatty acid compositions of the prior art makes the composition of the invention cost-effective to produce on a large scale (i.e., use of less amount of antioxidant) and highly tolerated, for example, even when administered to pediatric subjects.

Said compositions of the invention are capable of effectively and rapidly treating joint and/or muscle symptoms or disorders such as the diseases, symptoms or disorders listed from (i) to (vi) below in this description.

In addition, said compositions of the invention could also be capable of (i.i) protecting the gastric mucosa, (i.ii) treating diabetes, and (i.iii) treating diseases and/or disorders other than diabetes arising from or related to elevated blood glucose levels (regulation of blood glucose levels).

These purposes, and others that will become clear from the detailed description that follows, are achieved by the compositions, mixtures, and processes of the present invention through the technical features claimed in the united claims.

### BRIEF DESCRIPTION OF THE FIGURES

FIG. 1 shows the results of in vitro tests on the viability of human mesenchymal stem cells (hMSCs) after 2 weeks of treatment at three different concentrations of Lipocet®.

FIG. 2 shows the results of in vitro tests related to mineralization of hMSC after 18 days of treatment with a vitamin D, in the form of vitamin D3, administered as a product called UltraD3®, Lipocet® or with the mixture of the two, compared with the control sample (Ctrl).

FIG. 3 shows the results of in vitro tests related to mineralization of hMSC after, respectively, 18 days and 21 days of treatment with vitamin UltraD3®, or a mixture of vitamin named UltraD3® and Lipocet®, compared with the control sample (Ctrl).

FIG. 4 shows the Experimental Design.

FIG. 5 shows the evolution of body weight from the week before the intervention the intervention (W−1) to W17. Data are shown as mean±SD. N=8 rats for Groups 1, 2 and 4. N=9 rats for Group 3. N=9 rats and then N=8 rats from W5 for Group 5. Mixed effects analysis was performed on the data followed by Tukey's multiple comparison test. W−1 corresponds to D−1 and W0 corresponds to D+3.

FIG. 6 shows bone volume expressed in voxels versus bone volume at W0 for W4. Data are shown as mean±SD. A one-way ANOVA was performed followed by Tukey's multiple comparison test.

FIG. 7 shows the percentage of bone repair compared with the control group at (A) W4. Data are shown as mean±SD. A one-way ANOVA followed by Tukey's multiple comparison test was performed.

### DETAILED DESCRIPTION OF THE INVENTION

As a result of intensive research activity, the Applicant has developed innovative compositions and their uses in both therapeutic and nontherapeutic methods of treatment, as detailed in this description.

As an example, one of the formulations used in the experimental part involves Lipocet® 5 mg/ml+Vit. D (from Ultra D3) 0.001 mg/ml. For example, it should be kept in mind that in a hypothetical finished product there may be 1.6 g of Lipocet composition (sum of cetylated fatty acids+olive oil+antioxidant, e.g., TBHQ)+50 mcg of vitamin D3. UltraD3® has a vitamin D3 titer of about 0.2125%.

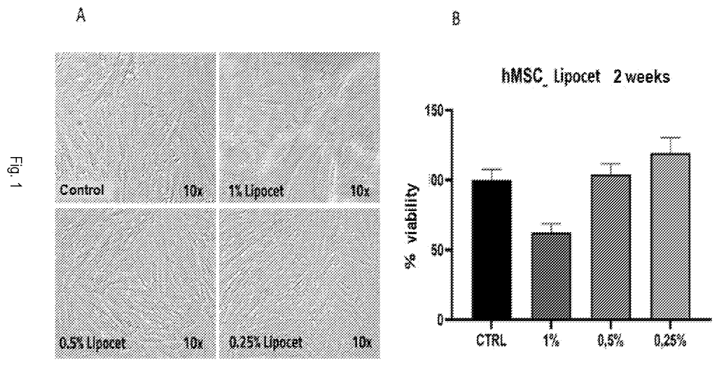

FIG. 1 shows the results of in vitro tests on the viability of human mesenchymal stem cells (hMSCs) after 2 weeks of treatment with three different concentrations (0.25%, 0.5%, 1%) of Lipocet®. Specifically, in FIG. 1A, left, are the microscopic photographs (10× magnification) of the cell colonies untreated (control sample, Ctrl) or supplemented with 1%, 0.5% or 0.25% Lipocet®, while in FIG. 1B, right, the histogram graph shows the % cell viability of the four samples tested after 2 weeks of treatment. With reference to FIG. 1, an amount of 5 mg/ml (0.5%) was chosen to conduct the subsequent experimental tests because it is the amount acceptable by the cells. In the experimental part, a product called Lipocet® in an amount of 5 mg/ml (0.5%) was used in combination with a vitamin D, in the form of vitamin D3, in an amount of 0.001 mg/ml, administered as a product called UltraD3®.

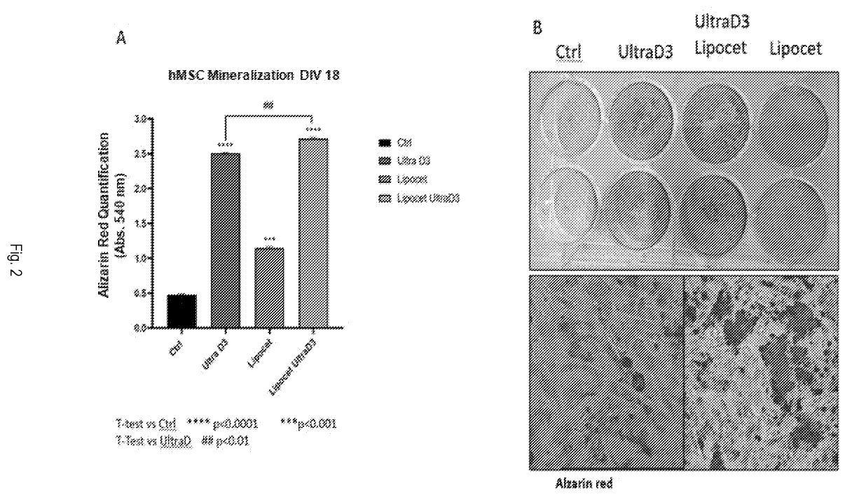

FIG. 2 shows the results of in vitro tests related to mineralization of hMSC after 18 days of treatment with a vitamin D, in the form of vitamin D3, administered as a product called UltraD3® (vitamin D3 0.001 mg/ml) Lipocet® (5 mg/ml, 0.5%) or with the mixture of the two (mixture according to the present invention), compared with the control sample (Ctrl). Specifically, calcium deposits were assessed by staining with Alizarin Red (Alizarin Red Staining Method-A simple and rapid screening method using alizarin red staining and ordinary optical microscopy to detect microcrystalline or noncrystalline calcium phosphate salts) both qualitatively (images-FIG. 2B) and quantitatively (histograms-FIG. 2A) by measuring absorbance at 540 nm.

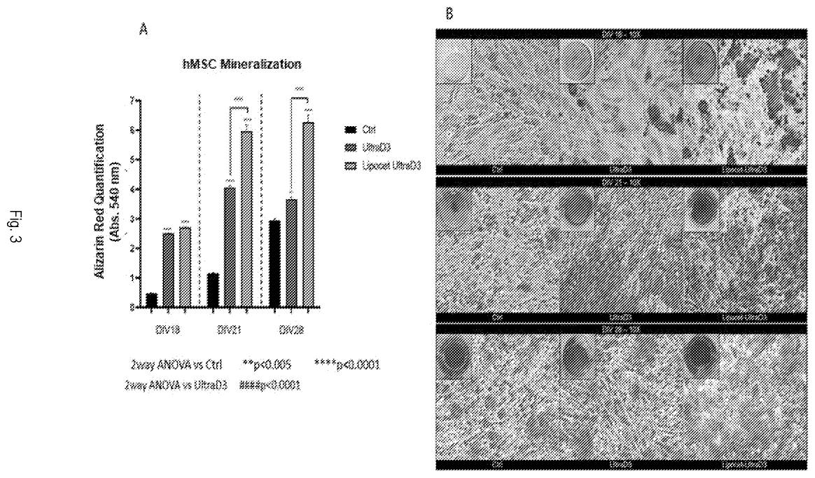

FIG. 3 shows the results of in vitro tests related to the mineralization of hMSCs after, respectively, 18 days and 21 days of treatment with vitamin UltraD3® (vitamin D3 supply of 0.001 mg/ml), or with a mixture of vitamin named UltraD3® and Lipocet® (mixture according to the present invention—0.001 mg/ml and 5 mg/ml, respectively), compared with the control sample (Ctrl). Specifically, mineralization was assessed by measurement of absorbance at 540 nm after staining by Alizarin Red, and the data are shown in terms of histogram in the graph on the left (FIG. 3A) and visually in the photos on the right (FIG. 3B) after 18 days, top photo, and after 21 days, bottom photo.

It is an object of the present invention a composition (in short, composition of the invention) comprising (I) and, optionally, (II), wherein:
(I) is a mixture (in short, mixture of the invention) comprising or, alternatively, consisting of:
(a) at least one cetylated fatty acid (or a mixture of cetylated fatty acids), wherein said fatty acid has a number of carbons in the range from C6 to C22, preferably from C8 to C20, more preferably from C12 to C18, even more preferably from C14 to C16, and may be saturated or unsaturated;
(b) at least one vitamin D, preferably at least one vitamin D3, and, optionally,
(c) at least one antioxidant in an amount by weight from 0.001% to 0.5% with respect to the total weight of the composition; and
(II) is at least one additive and/or excipient of pharmaceutical or food grade.

Preferably, said (a) at least one cetylated fatty acid is present in said composition comprising (I) and, optionally, (II) in an amount by weight from 25% to 99%, preferably in an amount by weight from 35% to 98%, more preferably in an amount from 45% to 95%, even more preferably in an amount by weight from 50% to 90%, with respect to the total weight of the composition.

Preferably, said (b) is present in said composition comprising (I) and, optionally, (II) in the form of at least one vitamin D, preferably in the form of at least one vitamin D3, more preferably in the form of at least one vitamin D3 carried within systems such as, for example, not limited to, micelles and liposomes, or aggregates, particularly liposomes or aggregates comprising or consisting of sucroester and lecithin and, optionally, starch as in the case of vitamin D3 present in the product called UltraD3® (see below).

Preferably, said (b) at least one vitamin D, preferably at least one vitamin D3, is present in an amount by weight from 25% to 99%, preferably in an amount by weight from 35% to 98%, more preferably in an amount by weight from 45% to 95%, even more preferably in an amount by weight from 50% to 90%, with respect to the total weight of the composition.

Preferably, said (a) at least one cetylated fatty acid and said (b) at least one vitamin D are present in said composition comprising (I) and, optionally, (II) in a weight ratio [(a):(b)] ranging from to 1:3 to 3:1, preferably ranging from 1:2 to 2:1, more preferably 1:1.

Preferably, said (c) at least one antioxidant is present in said composition comprising (I) and, optionally, (II) in an amount by weight from 0.001% to 0.5%, preferably from 0.005% to 0.1%, even more preferably from 0.01% to 0.08% with respect to the total weight of the composition.

Preferably, said (c) at least one antioxidant may be selected from the group comprising or, alternatively, consisting of: tert-butyl-hydroquinone (TBHQ), Aperoxid® TLA (as defined in the present invention), a mixture of tocopherols, a natural extract of rosemary, and mixtures thereof; more preferably (b) is tert-butyl-hydroquinone (TBHQ) and is present in an amount by weight from 0.001% to 0.5%, preferably from 0.005% to 0.1%, even more preferably from 0.01% to 0.08% by weight, with respect to the total weight of the composition. Preferably, said mixture (I) comprising (a)+(b)+(c) is present in said composition comprising (I) and, optionally, (II), in an amount from 50% to 98%, preferably from 60% to 95%, more preferably from 70% to 90%, by weight, with respect to the total weight of the composition.

Vitamin D, and in particular vitamin D3 (also known as cholecalciferol), of the present invention, is a vitamin D, or D3, either free or carried within structures known to the expert in the field, such as, for example, micelles and liposomes, of various natures. Said structures have, for example, the function of protecting the vitamin carried within them, increasing its solubility and stability in the formulation, and improving its absorption profile across biological membranes (e.g., dermis, gastro intestinal mucosa). According to a preferred aspect of the present invention, the vitamin D within the mixture (I) is vitamin D3 called UltraD3®, or sucrosomal vitamin D3, wherein the vitamin is enclosed or encapsulated or aggregated within a structure, primarily comprising at least one lecithin and at least one ester of a fatty acid carbohydrate, also referred to as sucroester, optionally, also in the presence of a starch (WO 2019/025922).

The composition of the invention can be a pharmaceutical composition, a medical device composition, a composition for a novel food (food) or supplement, or a cosmetic composition.

Preferably, said (a) at least one cetylated fatty acid (or a mixture of cetylated fatty acids) wherein said fatty acid has a number of carbons in the range from C6 to C22, preferably from C8 to C20, more preferably from C12 to C18, even more preferably from C14 to C16, and may be saturated or unsaturated, is a fatty acid esterified with cetyl alcohol (1-hexadecanol, CH₃(CH₂)₁₅OH).

Preferably, said (a) at least one cetylated fatty acid (or a mixture of cetylated fatty acids) is selected from the group comprising or, alternatively, consisting of: lauric acid, myristic acid, palmitic acid, palmitoleic acid, stearic acid, oleic acid, linoleic acid, eicosanoic acid, and mixtures thereof; more preferably said (a) comprises, or alternatively, consists of a mixture of cetylated myristic acid and cetylated oleic acid.

Even more preferably, said (a) at least one cetylated fatty acid (or a mixture of cetylated fatty acids) is a mixture that comprises or, alternatively, consists of a mixture of cetylated myristic acid, cetylated oleic acid, cetylated linoleic acid, and cetylated palmitic acid.

The composition of the invention comprising (I) and, optionally, (II) is for oral use, for example, in the form of a tablet, capsule, powder or granules.

Preferably, said (a) at least one cetylated fatty acid (or a mixture of cetylated fatty acids) that is present in said composition comprising (I) and, optionally, (II) has a fatty acid profile comprising or, alternatively, consisting of:
(a1) myristic acid: from 30% to 55%, preferably from 35% to 50%, more preferably about 41% by weight;
(a2) oleic acid: from 35% to 60%, preferably from 40% to 55%, more preferably about 46% by weight;
(a3) linoleic acid: from 3% to 12%, preferably from 5% to 10%, more preferably about 8% by weight;
(a4) palmitic acid: from 1% to 8%, preferably from 2% to 6%, more preferably about 3% by weight;
(a5-a9) various fatty acids (not myristoleic acid): from 1% to 4%, preferably from 1.5% to 3%, more preferably about 1.8% by weight;
   where % are % by weight, with respect to the total weight of fatty acids from a1 to a9.

For example, a mixture of cetylated fatty acids may have a profile like:
(a1) myristic acid about 41%, by weight;
(a2) oleic acid about 46% by weight;
(a3) linoleic acid about 8% by weight;
(a4) palmitic acid about 3 percent by weight;
(a5) stearic acid about 0.8% by weight;
(a6) cica palmitic acid 0.5 wt %;
(a7) eicosenoic acid about 0.3 wt %;
(a8) lauric acid about 0.2 wt %;
(a9) eicosanoic acid about 0.1 wt %.

An example of said (a) mixture of cetylated fatty acids is defined in Regulation (EU) 2022/187 of Feb. 10, 2022 and is on the market under the name Lipocet® (oral use formulation 1.6 g/day for adults).

Preferably, said (a) mixture of cetylated fatty acids is obtained by chemical synthesis (patent applications by the same Applicant) from cetyl alcohol, myristic acid, and oleic acid and is present in an amount by weight from 70% to 90%, more preferably from 75% to 85%, e.g., 83%-85% by weight, with respect to the total weight. Said mixture (a) may also comprise lower amounts of other of cetylated fatty acids such as, for example, palmitic acid and linoleic acid, which come from the esterification of fatty acids contained in the olive oil used in the composition. In said composition, the ester content ranges from 70% to 80%, of which cetyl oleate about 22%-30%, cetyl myristate about 41%-56%. Triglycerides about 22%-25%. Acidity index (mg KOH/g) less than or equal to 5. Saponification index (mg KOH/g) ranging from 130 to 150. Lipocet® formulation contains cetyl esters in an amount by weight from 70% to 80%, e.g. 76.5%, and a refined olive oil (food grade) in an amount by weight from 20% to 30%, e.g. 23.5%.

In an embodiment of the present invention, the Lipocet® formulation may be supplemented with (b) at least one vitamin D, preferably a vitamin D3 or UltraD3, and, optionally, with (c) at least one antioxidant, preferably a TBHQ.

Preferably, a qualitative and quantitative composition of the UltraD3 product may contain:
a vitamin D3 (100,000-120,000 IU/g cholecalciferol) preferably from 75% to 95% by weight, more preferably from 80% to 90%, such as 85%;
saccharic esters of fatty acids preferably from 5% to 25%, more preferably from 10% to 15%, e.g., 13% of type E473;
a natural starch, preferably a pregelatinized rice (Oryza sativa L.) starch, preferably from 0.1% to 3% by weight, more preferably from 0.5% to 2%, such as 1%;
a lecithin, preferably a sunflower lecithin, e.g., a lecithin supported on tricalcium phosphate and rice flour, preferably from 0.1% to 2%, more preferably from 0.5% to 1%, e.g., 1%.

In an embodiment, said (a) comprises a mixture of cetylated myristic acid and cetylated oleic acid with the provision that said (a) does not comprise cetylated myristoleic acid (it is of animal origin), particularly when the composition of the invention is for use in a method of treatment of osteoarthritis, joint pain, musculoskeletal pain including, but not limited to, those of an inflammatory nature.

In a further embodiment, said (a) comprises a mixture of cetylated myristic acid (it is of natural origin) and cetylated oleic acid which may additionally comprise cetylated myristoleic acid (it is of animal origin), particularly when the composition of the invention is for use in a method of treatment of protection of the gastric mucosa and regulation of blood glucose levels.

Myristic acid is tetradecanoic acid (CH₃(CH₂)₁₂COOH), a 14-carbon-atom saturated fatty acid found in milk derivatives (butter, cream, and cheese), coconut oil, palm kernel oil, and some spices (nutmeg). The myristic acid used in the present invention can be, for example, selected from those at 99% CAS 544-63-8 (EINECS 208-875-2) having a % composition (GLC): lauric acid C12:0 less than or equal to 1; myristic acid C14:0 greater than or equal to 99%; palmitic acid C16:0 less than or equal to 1.

Oleic acid is cis-9-octadecenoic acid (CH₃(CH₂)₇CHCH(CH₂)₇COOH), a monounsaturated carboxylic acid with 18 carbon atoms, and is the most abundant constituent of most vegetable oils.

The oleic acid used in the present invention can be, for example, selected from those having at least 78% of oleic acid CAS 112-80-1 (EINECS 204-007-1) with a % composition (GLC) for example: [lauric acid+myristic acid]C12:0+C14:0 less than or equal to 0.5; oleic acid C18:1 greater than or equal to 78%; linoleic acid C18:2 less than or equal to 15; and other C18:3 less than or equal to 1.

The cetyl alcohol (1-hexadecanol) used in the present invention can be, for example, selected from those having CAS 36653-82-4 (EINECS 253-149-0).

Advantageously, when said (a) comprises or consists of a mixture of cetylated myristic acid and cetylated oleic acid, the molar ratio between cetylated myristic acid and cetylated oleic acid is from 4:1 to 1:1, preferably it is from 3:1 to 1.5:1, more preferably it is (2.0±0.2):1.

In a preferred embodiment, the composition of the invention comprises the (I) mixture comprising or, alternatively, consisting of:
(a) a mixture comprising or, alternatively, consisting of a cetylated myristic acid and a cetylated oleic acid, preferably in a molar ratio from 4:1 to 1:1, preferably from 3:1 to 1.5:1, more preferably (2.0±0.2):1,
(b) at least one vitamin D, preferably a vitamin D3,
(c) at least one antioxidant, in an amount by weight from 0.001% to 0.5%, preferably from 0.005% to 0.1%, more preferably from 0.01% to 0.08%, with respect to the total weight of the composition.

Preferably, said (a) a mixture of cetylated fatty acids is present in said composition in an amount by weight from 25% to 99%, preferably in an amount by weight from 35% to 98%, more preferably in an amount from 45% to 95%, even more preferably in an amount by weight from 50% to 90%, with respect to the total weight of the composition.

Preferably, said (b) is present in said composition in the form of at least one vitamin D, preferably in the form of at least one vitamin D3, more preferably in the form of at least one vitamin D3 carried within systems such as, for example, not limited to, micelles and liposomes, or aggregates, particularly liposomes or aggregates comprising or consisting of sucroester and lecithin and, optionally, starch as in the case of vitamin D3 present in the product called UltraD3® (see below).

Preferably, said (b) at least one vitamin D, preferably at least one vitamin D3, is present in said composition in an amount by weight from 25% to 99%, preferably in an amount by weight from 35% to 98%, more preferably in an amount by weight from 45% to 95%, even more preferably in an amount by weight from 50% to 90%, with respect to the total weight of the composition.

Preferably, said (a) mixture of cetylated fatty acids and said (b) at least one vitamin D are present in said composition in a weight ratio [(a):(b)] ranging from 1:3 to 3:1, preferably ranging from 1:2 to 2:1, more preferably 1:1.

Preferably, said (c) at least one antioxidant is present in said composition in an amount by weight from 0.001% to 0.5%, preferably from 0.005% to 0.1%, even more preferably from 0.01% to 0.08% with respect to the total weight of the composition.

Preferably, said (c) at least one antioxidant may be selected from the group comprising or, alternatively, consisting of: tert-butyl-hydroquinone (TBHQ), Aperoxid® TLA (as defined in the present invention), a mixture of tocopherols, a natural extract of rosemary, and mixtures thereof; more preferably (b) is tert-butyl-hydroquinone (TBHQ) and is present in an amount by weight from 0.001% to 0.5%, preferably from 0.005% to 0.1%, even more preferably from 0.01% to 0.08% by weight, with respect to the total weight of the composition. Preferably, said mixture (I) comprising (a)+(b)+(c) is present in said composition comprising (I) and, optionally, (II), in an amount from 50% to 98%, preferably from 60% to 95%, more preferably from 70% to 90%, by weight, with respect to the total weight of the composition.

Preferably, said mixture (I) comprising (a)+(b)+(c) is present in said composition in an amount from 50% to 98%, preferably from 60% to 95%, more preferably from 70% to 90%, with respect to the total weight of the composition.

Antioxidant (c) is optionally included in the composition of the invention along with (a) and (b), and may be any antioxidant deemed by the technical expert in field to be suitable for use in pharmaceutical compositions, medical device compositions, novel foods (foods), supplements, or cosmetic compositions for the purposes stated in this description.

Antioxidants are often added to foods containing mostly fat to retard the development of rancidity that is initiated in the presence of oxygen. Natural antioxidants include flavonoids, polyphenols, ascorbic acid (vitamin C) and tocopherols (vitamin E). Synthetic antioxidants include butylhydroxyanisole (BHA), butylhydroxytoluene (BHT) and ethoxyquinoline.

Preferably, said (c) at least one antioxidant may be selected from the group comprising or, alternatively, consisting of: tert-butyl-hydroquinone (TBHQ), Aperoxid® TLA (as defined in the present invention), a mixture of tocopherols, a natural rosemary extract and mixtures thereof; more preferably (b) is tert-butyl-hydroquinone (TBHQ).

Tert-butylhydroquinone (TBHQ or tertiary butylhydroquinone) is used in the food industry as a preservative for unsaturated vegetable oils and many edible animal fats. TBHQ is authorized as a food additive in the European Union with an acceptable daily intake (ADI) of 0.7 mg/kg body weight and is identified by code E319.

Aperoxid® TLA (registered trademark) is the trade name of an antioxidant effective in counteracting fat rancidity, used for example in cosmetics. Aperoxid® TLA is a mixture comprising tocopherol ((±)-α-tocopherol) 10-15%, lecithin >50%, ascorbyl palmitate 8-10% (an ester formed from ascorbic acid and palmitic acid that creates a fat-soluble form of vitamin C) and citric acid <1% (INCI NAME: lecithin, tocopherol ((±)-α-tocopherol), ascorbyl palmitate, citric acid; CAS NUMBER: 8002-43-5, 10191-41-0, 137-66-6, 77-92-9).

In a preferred embodiment, the composition of the invention comprises the (I) mixture comprising or, alternatively, consisting of:
(a) a mixture of cetylated myristic acid and cetylated oleic acid, preferably in the molar ratio of 4:1-1:1 or 3:1-1.5:1 or (2.0+0.2):1,
(b) vitamin D3, preferably vitamin UltraD3®, in 0.001 mg/MI vitamin D3 and, optionally,
(c) tert-butyl-hydroquinone (TBHQ), in an amount 0.001%-0.5% or 0.005%-0.1% or 0.01%-0.08% by weight with respect to the total weight of the composition.

In a more preferred embodiment, the composition of the invention comprises the (I) mixture comprising or, alternatively, consisting of:
(a) a mixture of cetylated myristic acid and cetylated oleic acid in the molar ratio of 3:1-1.5:1, preferably (2.0±0.2):1,
(b) an UltraD3 vitamin, 0.001 mg/ml vitamin D3, and
(c) a tert-butyl-hydroquinone (TBHQ) in an amount from 0.01% to 0.08% by weight with respect to the total weight of the composition.

In a further embodiment, the (I) mixture of the invention, in addition to (a), (b) and, optionally, (c), also comprises (d) a vegetable oil.

Preferably said (d) vegetable oil is selected in the group comprising or, alternatively, consisting of olive oil, high oleic acid sunflower oil, any high oleic acid vegetable oil and mixtures thereof. Advantageously, (d) is olive oil.

In a preferred embodiment, the (I) mixture of the invention comprises or, alternatively, consists of:
said (a) at least one cetylated fatty acid (or a mixture of cetylated fatty acids), preferably a mixture of cetylated myristic acid and cetylated oleic acid, in a weight % from 50% to 90% with respect to the total weight of the composition, preferably from 60% to 85%, more preferably from 70% to 80%;
said (b) vitamin D, preferably vitamin D3, even more preferably vitamin D3 carried within systems such as, for example but not limited to, nanosomes and/or liposomes and/or aggregates, particularly liposomes or aggregates consisting of sucroester and lecithin as in the case of vitamin UltraD3® in an amount 0.001 mg/mL of vitamin D3;
said (c) at least one antioxidant, preferably tert-butyl-hydroquinone (TBHQ), in a weight % from 0.001% to 0.5% with respect to the total weight of the composition, preferably from 0.005% to 0.1%, more preferably from 0.01% to 0.08%; and
said (d) vegetable oil, preferably olive oil, in a % by weight from 9.5% to 50% with respect to the total weight of the composition, preferably from 14.5% to 40%, more preferably from 19.5% to 30%.

In a preferred embodiment, the (I) mixture of the invention comprises or, alternatively, consists of:
(a) comprising or consisting of a mixture of cetylated myristic acid and cetylated oleic acid in a molar ratio of 4:1-1:1 or 3:1-1.5:1, preferably (2.0+0.2):1; preferably in a weight % from 60% to 85% with respect to the total weight of the composition, preferably from 70% to 80%;
(b) 0.001 mg/ml of vitamin UltraD3;
(c) tert-butyl-hydroquinone (TBHQ) in a weight % from 0.005% to 0.1% with respect to the total weight of the composition, preferably from 0.01% to 0.08%; and
(d) olive oil in a % by weight from 15% to 40% with respect to the total weight of the composition, preferably from 19.5% to 30%.

In a more preferred embodiment, the (I) mixture of the invention comprises or, alternatively, consists of:
(a) comprising or consisting of a mixture of cetylated myristic acid and cetylated oleic acid in a molar ratio of (2.0±0.2):1 and in a weight % from 70% to 80%;
(b) vitamin UltraD3® 0.001 mg/ml of vitamin D3,
(c) tert-butyl-hydroquinone (TBHQ) in a weight % from 0.01% to 0.08% with respect to the total weight of the composition; and
(d) olive oil in a % by weight from 15% to 40% with respect the total weight of the composition, preferably from 19.5% to 30%.

It is an object of the present invention the compositions of the invention set forth in this description comprising: (I) the mixture of the invention, comprising or consisting of (a) and (b) and, optionally, (c) and/or (d) (as defined above), and, optionally, (II) at least one additive and/or excipient of pharmaceutical or food grade, said compositions being for use as a medicament when administered to a subject in need.

It is an object of the present invention the compositions of the invention comprising (a) and (b) and, optionally, (c) and/or (d) (according to all of embodiments described in this description) for use in a method of treatment, preventive and/or curative and/or symptomatic, of diseases, symptoms or disorders such as:
(iv) post-traumatic osteoarticular and musculoskeletal diseases including sports injuries;
(v) degenerative joint diseases, preferably arthrosis, gonarthrosis, coxarthrosis, and/or
(vi) Inflammatory-traumatic tendon and muscle forms, when administered to a subject in need.

It is an object of the present invention the compositions of the invention comprising (a) and (b), and, optionally, (c) and/or (d) (according to all of embodiments described in this description) (vii) for use in bone proliferation-regeneration, particularly for use in a method of promoting and improving bone proliferation-regeneration.

It is an object of the present invention the compositions of the present invention comprising (a) and (b) and, optionally, (c) and/or (d) (according to all of embodiments described in this description) (viii) for use in a method of treatment, preferably preventive, of osteoporosis, bone fracture, prosthetic surgery, bone growth, osteoarthritis, consolidation post-fracture, joint pain, musculoskeletal pain, including, but not limited to, those of an inflammatory nature.

It is an object of the present invention the compositions of the invention comprising (a) and (b) and, optionally, (c) and/or (d) (according to all of embodiments described in this description) (ix) for use in a method of treatment of gastric mucosal protection and regulation of blood glucose levels.

Preferably, compositions of the invention for use in a method of treatment of diseases, symptoms or disorders from (iv) to (ix) as identified above comprise the (I) mixture comprising or, alternatively, consisting of:
(a) comprising or consisting of a mixture of cetylated myristic acid and cetylated oleic acid in the molar ratio of 3:1-1.5:1, preferably (2.0±0.2):1,
(b) vitamin UltraD3® in 0.001 mg/ml of vitamin D3 and, optionally,
(c) tert-butyl-hydroquinone (TBHQ) in an amount of 0.01%-0.08% by weight with respect to the total weight of the composition.

Preferably, the compositions of the invention for use in a method of treatment of diseases or disorders from (iv) to (ix) as identified above comprise the (I) mixture comprising or, alternatively, consisting of:
(a) a mixture of cetylated myristic acid and cetylated oleic acid in a molar ratio of (2.0±0.2):1 and in a weight % from 70% to 80%;
(b) a vitamin UltraD3® 0.001 mg/ml of vitamin D3
(c) a tert-butyl-hydroquinone (TBHQ) in a weight % from 0.01% to 0.08% with respect to the total weight of the composition; and, optionally,
(d) an olive oil in a % by weight from 15% to 40% with respect to the total weight of the composition, preferably from 19.5% to 30%.

Said compositions of the invention for use in a method of treatment of the diseases, symptoms or disorders listed from (iv) to (vi), as defined above, are administered to said subject preferably topically or transdermally; more preferably topically.

Advantageously, the cetylated fatty acids included in mixture (a) have a molecular weight range from 420 Da to 510 Da. This molecular weight range enables mixture (a), and thus the composition of the present invention, to effectively cross the dermal barrier (skin) facilitating topical absorption.

Alternatively, said compositions of the invention for use in a method for the treatment of diseases, symptoms or disorders listed from (iv) to (vi), as defined above, are administered to said subject preferably orally.

It is an object of the present invention the compositions of the invention comprising (a) and (b) and, optionally, (c) and/or (d) (according to all of embodiments described in this description) for use in a method of treatment, preventive and/or curative and/or symptomatic, of:
(i.i) symptoms and/or disorders arising from or related to damage to the gastric mucosa (gastric mucosal protection treatment),
(i.ii) diabetes and disorders or symptoms arising from or related to diabetes,
(i.iii) diseases and/or disorders other than diabetes resulting from or related to high blood glucose levels, when administered to a subject in need.

Said (ii.) symptoms or disorders arising from or related to damage to the gastric mucosa (protection of the gastric mucosa) are selected from the group comprising or, alternatively, consisting of: gastric ulcer, gastroesophageal reflux disease (GERD), and gastric pyrosis.

Said (i.iii) diseases and/or disorders other than diabetes arising from or related to elevated blood glucose levels are selected from the group comprising or, alternatively, consisting of: hyperglycemia, chronic hepatopathy, obesity.

Said compositions of the invention for use in a method of treatment of the diseases, symptoms or disorders listed from (i) to (ix) and from (i.i) to (i.iii), as defined above, are administered to said subject preferably orally.

Preferably, the compositions of the invention for use in a method of treatment of diseases or disorders from (iv) to (ix) and from (i.i) to (i.iii) as identified above comprise the (I) mixture comprising or, alternatively, consisting of:
(a) a mixture of cetylated myristic acid and cetylated oleic acid, preferably in the molar ratio of 4:1-1:1 or 3:1-1.5:1 or preferably (2.0±0.2):1,
(b) a UltraD3® vitamin, preferably in an amount 0.001 mg/ml of vitamin D3, and, optionally,
(c) a tert-butyl-hydroquinone (TBHQ) in 0.01%-0.08% by weight with respect to the total weight of the composition.

The compositions of the invention can be formulated in a liquid form, such as solution, emulsion, suspension, oral gel, syrup, spray, oil or in a semisolid form, such as oral gel, softgel, or in a solid form, such as powder, powder obtained by spray drying, granules, microgranules, flakes, aggregates, orosoluble sticks, tablets, effervescent tablets, capsules, suppositories, bars and equivalent forms known to the expert in the field.

Advantageously, when the composition of the invention is for oral use it is formulated in solid or liquid form, more preferably in the form of oil or powder or powder obtained by spray drying or is in the form of spray or drink or food.

For example, the composition of the invention may be formulated for oral use as capsules comprising (a) the mixture of cetylated fatty acids according to the present invention, (b) vitamin D, preferably vitamin D3, even more preferably vitamin UltraD3®, optionally (c) at least one antioxidant as previously defined and excipients such as, for example but not limited to, gelatin, glycerol, and/or preservatives. According to one aspect of the present invention, the composition is in a gelatin capsule form, e.g., bovine gelatin from 100 mg to 500 mg, e.g., 200 mg, or 300 mg, or 400 mg.

It is an object of the present invention methods for the treatment of diseases, symptoms or disorders listed above from (i) to (ix) and from (i.i) to (i.iii), preferably by topical or transdermal administration, or, alternatively, for the treatment of diseases, symptoms or disorders listed above from (i) to (ix) and from (i.(i) to (i.iii), preferably by oral administration, to a subject in need of an effective amount of one of the compositions of the invention comprising (a) and (b) or (a) and (b) and (c) and/or (d) described herein.

The appropriate dosage of the composition of the present invention will depend, for example, on the condition to be treated/prevented, the severity and course of the condition, whether the composition is administered for preventive or therapeutic purposes, previous therapy, patient's medical history, and response to the composition and the discretion of the treating physician.

The composition of the invention is administered orally, the amount of the composition administered can range from 10 mg to 30 mg depending on the body weight of the treated subject, for example, for a subject weighing about 70 kg, an amount equal to about 30 mg of said (a) mixture of cetylated acids is administered.

The composition of the invention is appropriately administered to the subject at once or over a series of treatments.

The composition may be administered as a sole treatment or in combination with other compositions or therapies (i.e., with adjuvant action) useful in the treatment, preventive and/or curative and/or symptomatic, of the diseases, symptoms and/or disorders listed from (i) to (ix) and/or listed from (i.i) to (i.iii) as described in this description.

It is an object of the present invention the non-therapeutic use of the composition of the invention comprising (a) and (b) or (a) and (b) and/or (c) and/or (d) for the protection of the gastric mucosa, wherein said composition is preferably formulated for oral use.

The composition of the invention protects the gastric mucosa in such a way as to prevent damage to the gastric mucosa and thus the occurrence of disorders related to the gastric area such as, for example, gastric ulcer, gastroesophageal reflux disease (GERD), and gastric pyrosis.

The term “subjects” in the context of the present invention refers to human subjects or animal subjects (e.g., companion animals such as dogs or cats or other mammals). Preferably, the compositions of the invention are for use in methods of treatment of human subjects.

“Treatment method” in the context of the present invention means an intervention, comprising the administration of a substance, or mixture of substances, or combination thereof, having as its purpose the elimination, reduction/diminution, or prevention of a pathology or disease and its symptoms or disorders.

The term “medical device” in the context of the present invention is used in the meaning according to Italian Legislative Decree Feb. 24, 1997, no. 46 (or according to the new Medical Devices Regulation (EU) 2017/745 (MDR)), i.e., it means a substance or other product, used alone or in combination, intended by the manufacturer to be used in humans for the purpose of diagnosis, prevention, control, therapy, or mitigation of a disease, which product does not exert its principal action, in or on the human body, for which it is intended, by pharmacological or immunological means or by metabolic process but whose function may be assisted by such means.

Compositions of the invention optionally comprise (II) at least one additive and/or excipient of pharmaceutical or food grade, such as a substance without therapeutic activity suitable for pharmaceutical or food use, such as, for example, diluents, solvents (e.g. water, glycerin, ethyl alcohol), solubilizers, thickeners, sweeteners, anti-caking agents, flavoring agents, coloring agents, lubricants, surfactants, antimicrobials, antioxidants, preservatives, pH-stabilizing buffers, acidulants, and all auxiliary substances known to the expert in the field.

The compositions of the invention, in addition to said (a), (b), and, optionally, (c) and/or (d) may also include other active components such as, for example, anti-inflammatories, probiotics, antacids, products for the treatment of joint and/or muscle disorders, vitamins of the group B and E, mineral salts, pain relievers, folic acid or folate, menthol, glucosamine, chondroitin, methylsulfonylmethane (MSM), and essential oils.

Unless otherwise stated, a statement that a composition “comprises” one or more components or substances means that other components or substances may be present in addition to the one, or those, specifically stated. Unless otherwise specified, the expression composition comprises a component in an amount “within a range from x to y” means that said component may be present in the composition in all amounts in said range, even if not made explicit, extremes of the range included.

According to a preferred aspect of the present invention, said (a), at least one cetylated fatty acid or mixture of cetylated fatty acids is produced according to the process described in patent IT201900007311 in the name of the same Applicant.

In general, said process comprises the steps of:
(I) bring into contact at least one fatty acid, cetyl alcohol and a metal catalyst in a reactor chamber in the absence of solvent, to give a reaction mixture; followed by
(II) saturate the container with an inert gas by bringing the container to pressure P1 of about 1 atmosphere, apply a flow of said inert gas through said chamber; followed by
(III) apply an initial heating ramp to said reaction mixture until a temperature T1 from 120° C. to 200° C. is reached at a pressure P1 of about 1 atmosphere and in the presence of inert gas flow to initiate an esterification reaction with initial formation of cetylated said fatty acid or mixture of cetylated fatty acid and esterification water; followed by
(IV) keep said reaction mixture stirring at said temperature T1 and pressure P1 for a time from 10 minutes to 2 hours; followed by
(V) apply a second heating ramp to said reaction mixture until it reaches a temperature T2 ranging from 201° C. to 260° C. at a pressure P1 of about 1 atmosphere and in the presence of inert gas flow to give advancement of the esterification reaction with further formation of said cetylated fatty acid or mixture of cetylated fatty acid and esterification water; followed by
(VI) keep said reaction mixture stirring at said temperature T2 and pressure P1 and in the presence of inert gas flow for a time ranging from 4 hours to 12 hours;
(VII) apply a vacuum program in the chamber that reduces the reaction pressure until a reduced pressure P2 from 100 mbar to 10 mbar is reached;
(VIII) maintain said reaction mixture under stirring at said reduced pressure P2 and in the presence of inert gas flow for from 30 minutes to 4 hours to give complete formation of said at least one crude cetylated fatty acid or crude cetylated fatty acid mixture (MI).

Said steps may then be followed by a step (IX) of filtration, e.g., on decolorizing earth and/or filtering earth in a filter press to obtain at least one filtered cetylated fatty acid (Mf), and a step (X) of deodorization carried out, e.g, with a treatment of said at least one filtered cetylated fatty acid (Mf) in a reactor at a temperature T3 ranging from 150° C. to 200° C. and a reduced pressure P3 ranging from 1 mbar to 20 mbar in the presence of a water steam flow for a period of time ranging from 1 hour to 5 hours to give at least one refined cetylated fatty acid (MF).

It is an object of the present invention a process for preparing a composition of the invention comprising said (a) at least one cetylated fatty acid (or mixture of cetylated fatty acid) as defined in the present description, wherein said process comprises the step (XI) of mixing said at least one filtered cetylated fatty acid (Mf), obtainable according to the previously described process, with (b) vitamin D, preferably vitamin D3, even more preferably vitamin UltraD3®, wherein said (b) is mixed in a weight % of 0.0001% (0.001 mg/ml). According to a preferred aspect of the present invention to the mixture of (a) and (b) is also added (c) at least one antioxidant (as defined in the present invention), wherein said (c) is mixed in a weight % from 0.001% to 0.5% with respect to the total weight of the composition (composition of the invention), preferably from 0.005% to 0.1%, more preferably from 0.01% to 0.08%. Please note that patent documents WO2004/084829 A1 and WO2017/029580 A1 are to be considered as “incorporated by reference” in this description.

In a preferred embodiment, said process for preparing a composition of the invention comprises the step of mixing said at least one filtered cetylated fatty acid (Mf) or at least one refined cetylated fatty acid (MF) comprising or, alternatively, consisting of a mixture of cetylated myristic acid and cetylated oleic acid, preferably in a molar ratio of 4:1-1:1 or 3:1-1.5:1 or (2.0+0.2):1, and in which said (b) vitamin D is UltraD3® vitamin in a % by weight of 0.0001% (0.001 mg/ml), and in which said optional component (c) at least one antioxidant is tert-butyl-hydroquinone (TBHQ) in a weight % from 0.001% to 0.5% with respect to the total weight of the composition, preferably from 0.005% to 0.1%, more preferably from 0.01% to 0.08%.

In an embodiment, said at least one filtered cetylated fatty acid (or mixture of cetylated fatty acid) (Mf), preferably a mixture of filtered cetylated myristic acid and cetylated oleic acid, prior to undergoing step (X) of deodorization, is fortified with said (d) vegetable oil (as defined in the present invention), preferably olive oil.

In an embodiment, prior to step (X) of deodorization, said at least one filtered cetylated fatty acid (Mf), preferably a mixture of filtered cetylated myristic acid and cetylated oleic acid, is subjected to step (XII) of blending said (Mf) with said (d) vegetable oil, preferably olive oil, to form a mixture comprising (Mf) and (d) which is subsequently subjected to step (X) of deodorization.

In a preferred embodiment, step (X) of deodorization of the previously described process to give at least one refined cetylated fatty acid (MF), preferably a mixture of refined cetylated myristic acid and cetylated oleic acid, and step (XI) of mixing said at least one refined cetylated fatty acid (MF) with vitamin D preferably vitamin D3, even more preferably vitamin UltraD3® and, optionally, at least one antioxidant, preferably TBHQ, and, optionally, step (XII) of blending the at least one filtered cetylated fatty acid (Mf) with (d) vegetable oil, are carried out in the same reactor.

Advantageously, the composition object of the present invention, shown in Experimental Part II as reference samples with a vitamin D, preferably a vitamin D3, even more preferably a vitamin called Ultra D3, administered orally is able to improve bone healing, preferably long bone healing, after four weeks. Advantageously, the composition of the present invention, represents a therapeutic agent to promote bone healing when administered early after fracture, during the inflammation/hematoma phase.

An embodiment of the composition object of the present invention, referred to in Experimental Part II as reference samples with vitamin D is given below.

Reference samples with a vitamin D (object of the present invention), preferably a vitamin D3, even more preferably a vitamin called Ultra D3, contain a lipid-based compound. The lipid-based compound is a mixture of cetylated fatty acids called Lipocet.

The mixture of cetylated fatty acids called Lipocet comprises cetylated myristic acid and cetylated oleic acid.

The reference sample with vitamin D (object of the present invention) contains a lipid-based compound, which is a mixture of cetylated fatty acids called Lipocet, in the presence of vitamin D3.

Vitamin D3 can be as such, or it can be in the form of a vitamin D3 processed with other ingredients to give a vitamin called Ultra D3 that comprises: a vitamin D3, sucroester, e.g. (E473), a vegetable lecithin, e.g. a sunflower lecithin, e.g. E322, and a starch, e.g. a rice starch, e.g. a pregelatinized rice starch.

An example of a commercially available vitamin called Ultra D3 comprises: vitamin D3 100 SD/S (cholecalciferol, alpha-tocopherol (E307), sodium ascorbate (E301), silicon dioxide (E 551), sodium starch octenylsuccinate (E 1450), medium-chain triglycerides, sucrose esters of fatty acids (E473), pregelatinized rice starch (Oryza sativa), sunflower lecithin (E322) [tricalcium phosphate (E341), rice flour]. Sucroester is also known as sucrose esters of fatty acids. Vitamin D3: 72,000-100,000 IU/g (=2,500 μg cholecalciferol/g).

An example of a reference sample with vitamin D (object of the present invention) containing a lipid-based compound, a mixture of cetylated fatty acids called Lipocet, in the presence of vitamin D3, may comprise: Ingredients: Purified water; Glycerol (E 422); cetylated myristic acid, cetylated oleic acid, myristic acid and oleic acid; Sucrose esters of fatty acids (E 473); Thickener: Xanthan gum (E 415); flavors; Cholecalciferol [Vitamin D3, alpha-tocopherol (E 307), sodium ascorbate (E 301), silicon dioxide (E 551), starch-sodium octenyl succinate (E 1450), medium-chain triglycerides, sucrose esters of fatty acids (E 473), pregelatinized rice starch (Oryza sativa L.), Lecithins (E 322), tricalcium phosphate (E 341(iii)), rice flour); preservatives: potassium sorbate (E 202) and sodium benzoate (E 211); acidifier: citric acid (E 330); antioxidant: rosemary extracts (E 392) [sunflower seed oil]; sweetener: sucralose (E 955). An example of this reference sample with vitamin D includes Lipocet (mixture of cetylated fatty acids) e.g., 8.160 mg and vitamin D3, e.g., 0.5 mg, in 100 ml (1 sachet 816.000 mg and 50.000 mcg equal to 2000 I.U.).

### Experimental Part I

By MTT assay, in vitro cell viability was assessed on human mesenchymal stem cells (hMSCs) following treatment with three different concentrations (0.25%, 0.5%, 1%) of Lipocet® compared with untreated hMSC cell culture (control sample). The optimal percentage selected for the subsequent experimental steps was 0.5% Lipocet® corresponding to 5 mg/ml. Next, treatment-induced mineralization (lasting 18 days) of hMSCs was evaluated with: Lipocet® (5 mg/ml, 0.5%), UltraD3® (0.001 mg/ml), Lipocet® (5 mg/ml, 0.5%) UltraD3® (0.001 mg/ml) mixture, and control treatment. Quantification was done following staining with Alizarin Red by reading the absorbance of each sample at 540 nm.

The previous test was repeated by comparing the treatment of hMSCs, performed for 18 days and 21 consecutive days, with vitamin UltraD3® (0.001 mg/ml) and a mixture of vitamin UltraD3® and Lipocet® (mixture according to the present invention—0.001 mg/ml and 5 mg/ml, respectively), against the control sample (Ctrl). Mineralization was then assessed by measurement of absorbance at 540 nm after Alizarin Red staining.

### Experimental Part II

### In Vivo Proof-of-Concept Study of a Lipid-Based Compound in a Rat Femur Critical Size Bone Defect Model

### Materials and Methods

This study was conducted on 40 adult male rats divided into five experimental groups, described below. Surgery for critical size femoral bone defect (CSBD; 5 mm RatFix System RISystem, Switzerland) was performed on the left femur to evaluate the healing of the bone defect after oral administration of:
reference samples, and
reference samples with a vitamin D (object of the present invention), preferably a vitamin D3, even more preferably a vitamin called Ultra D3.

The reference sample contains a lipid-based compound. The lipid-based compound is a mixture of cetylated fatty acids called Lipocet.

The mixture of cetylated fatty acids called Lipocet comprises cetylated myristic acid and cetylated oleic acid.

The reference sample with vitamin D (object of the present invention) contains a lipid-based compound, which is a mixture of cetylated fatty acids called Lipocet, in the presence of vitamin D3.

Vitamin D3 can be as such, or it can be in the form of a vitamin D3 processed with other ingredients to give a vitamin called Ultra D3 that comprises: a vitamin D3, sucroester, e.g. (E473), a vegetable lecithin, e.g. a sunflower lecithin, e.g. E322, and a starch, e.g. a rice starch, e.g. a pregelatinized rice starch.

An example of a commercially available vitamin called Ultra D3 comprises: vitamin D3 100 SD/S (cholecalciferol, alpha-tocopherol (E307), sodium ascorbate (E301), silicon dioxide (E 551), sodium starch octenylsuccinate (E 1450), medium-chain triglycerides, sucrose esters of fatty acids (E473), pregelatinized rice starch (Oryza sativa), sunflower lecithin (E322) [tricalcium phosphate (E341), rice flour]. Sucroester is also known as sucrose esters of fatty acids. Vitamin D3: 72,000-100,000 IU/g (=2,500 μg cholecalciferol/g).

An example of a reference sample with vitamin D (object of the present invention) containing a lipid-based compound, a mixture of cetylated fatty acids called Lipocet, in the presence of vitamin D3, may comprise: Ingredients: Purified water; Glycerol (E 422); cetylated myristic acid, cetylated oleic acid, myristic acid and oleic acid; Sucrose esters of fatty acids (E 473); Thickener: Xanthan gum (E 415); flavoring; Cholecalciferol [Vitamin D3, alpha-tocopherol (E 307), sodium ascorbate (E 301), silicon dioxide (E 551), starch-sodium octenyl succinate (E 1450), medium-chain triglycerides, sucrose esters of fatty acids (E 473), pregelatinized rice starch (Oryza sativa L.), Lecithins (E 322), tricalcium phosphate (E 341(iii)), rice flour); preservatives: potassium sorbate (E 202) and sodium benzoate (E 211); acidifier: citric acid (E 330); antioxidant: rosemary extracts (E 392) [sunflower seed oil]; sweetener: sucralose (E 955). An example of this reference sample with vitamin D comprises Lipocet (mixture of cetylated fatty acids) e.g., 8.160 mg and vitamin D3, e.g., 0.5 mg, in 100 ml (1 sachet 816.000 mg and 50.000 mcg equal to 2000 I.U.).

Experimental Groups (n=8/group):
Group 1: Control—Reference object (tap water)
Group 2: high-dose test item 1 (lipid-based compound at 200 mg/kg BW)
Group 3: low-dose test item 1 (lipid-based compound at 100 mg/kg BW)
Group 4: high-dose test item 2 (lipid-based compound at 200 mg/kg BW with Ultra vitamin D at 6.24 μg/kg BW)
Group 5: low-dose test item 2 (lipid-based compound at 100 mg/kg BW with Ultra vitamin D at 3.12 μg/kg BW). BW=Body Weight.

### Experimental Design:

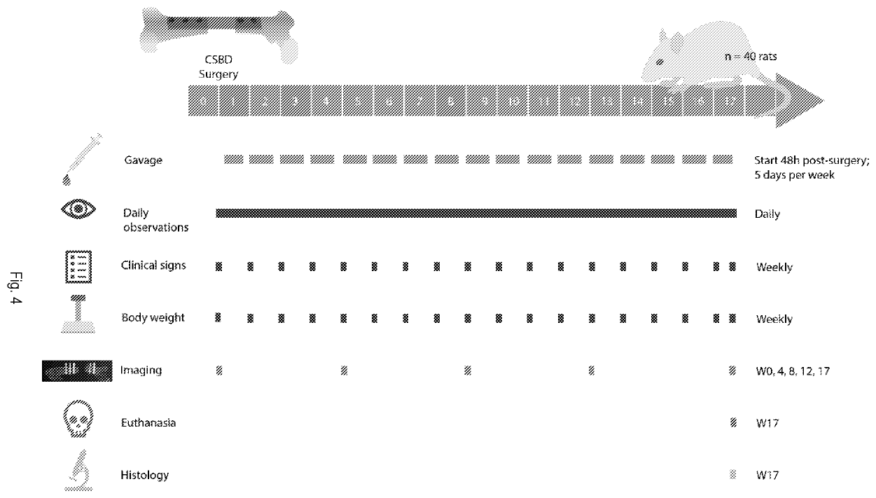

The experimental design is summarized in FIG. 4.
Acclimatization for a minimum period of five days
Surgery: W0; CSBD surgery (groups 1 to 5+4 reserve animals; n=44)
Life stage:
Oral gavage for five days a week with the reference item (group 1) or test items (groups 2 to 5) (n=40)
Postsurgery follow-up at least three days after surgery (n=42)
Daily observation (mortality and morbidity) (once daily) (n=44, then n=40)
Clinical signs (every day in the first three days after surgery, then once a week) (n=44, then n=40)
Body weight (BW) (every day in the first three days after surgery and once a week) (n=44, then n=40)
Food and water visual checks (daily) (n=44, then n=40)
Follow-up μCT at W0-W1 (n=44), W4, W8, W12 and W17 (n=40)
   Euthanasia (17 weeks after surgery) and subsequent analysis:
   Euthanasia (W17; n=40)
      Withdrawal of the operated left femur and fixation in formolus
      Histological analysis
   Evaluation of bone healing with CSBD:
      μCT on left femur
      W0-W1 (n=44), W4, W8, W12 and W17 (n=40)
      Histology
         at W17 (n=40)
         region of the bone defect
         Toluidine blue staining
         Microscopic analysis and scoring of the bone healing process

### Experiment in Life

### Surgery

The surgery was performed according to SOP ANIM37 by ERC trained staff on the left leg. As a first step, the surgery site (anterolateral and postero-lateral side of the left thigh) was shaved. Briefly, a lateral longitudinal skin incision was made on the left leg, and the femur was exposed by blunt dissection of the fascia between the quadriceps and hamstring muscles. The femur was then exposed for deformation. After local administration of an analgesic on the left femur, a 5-mm bone defect was induced according to the following procedure:
1. Placement of the plate along the bone surface, introducing the hip side first;
2. Maintenance of the plate next to the bone with two forceps+identification of the eight holes;
3. Drill hole No. 2 and insert the dedicated screw,
4. The head of screw No. 2 was kept intact because it was used as a guide for CSBD induction;
5. Drilling of hole No. 7 and insertion of the dedicated screw. The head of screw No. 7 was removed because it was not being used for the guide that allows induction of the CSBD;
6. Drill the other holes in this way: 1-3-6. The heads of screws 1 and 3 were removed as they were not used for the guide that allows induction of the CSBD, but the head of screw 6 was retained;
7. Place the cutting wires under the femur, one on the left side and the other on the right side;
8. Place the guide with the help of the remaining screw heads;
9. Place the two cutting wires in the grooves of the dedicated guide;
10. Bone cutting to induce CSBD;
11. Removal of the bone piece.

### Postsurgery Follow-Up

After surgery, sterile physiological serum (500 μl) was injected subcutaneously between the shoulders before the animals recovered from anesthesia. The animals were maintained alone in a 37° C. incubator until complete recovery. Each rat was monitored closely and frequently for at least 30 min and until complete recovery from anesthesia. Subsequently, they were housed with their cage mate.

Postsurgery follow-up was performed as follows:
Antibiotic treatment was given before surgery (cefazolin; 250 mg/kg) and for two days after surgery;
Buprenorphine (10 μg/kg) was injected twice daily for two days after surgery;
To facilitate their recovery, surgery-specific nutrient hydrogels (SAFE® gel diet energy—SAFE Diets) were placed in the cage with free access to this nutrition for 48 hours after surgery;
The rats were monitored daily for recovery of locomotion, normal behavior, body weight measurement, and presence of any signs of infection.

### Test Preparation and Oral Administration

The test product was administered daily per os by gavage with a feeding tube (5 days a week). Test administration began on the Monday following the week of surgery to provide the same treatment doses to all animals. Thus, administration occurred five days a week, Monday through Friday, for 16 weeks. The control group (Group 1) was given the reference item (i.e., tap water) to simulate animal handling and oral gavage gesture.

The volume of the test object was determined according to the animal's body weight and group as follows:
Group 1: Control; 252 μl of reference item (tap water)/100 g BW per day (5 days per week)
Group 2: high-dose test item 1; 252 μl of test item 1/100 g BW per day (5 days per week)
Group 3: low-dose test item 1; 126 μl of test item 1/100 g BW per day (5 days per week)
Group 4: high-dose test item 2; 252 μl of test item 2/100 g body weight per day (5 days per week)
Group 5: low-dose test item 2; 126 μl of test item 2/100 g BW per day (5 days per week)

### Microcomputed Tomography (μCT)

Scanning imaging (3D μCT acquisition) was first performed on all rats (n=44), up to seven days after surgery (i.e., at W0-W1) to confirm plaque maintenance and CBSD establishment. Then, μCT imaging was performed at W4, W8, W12 and W17 after surgery on all rats (n=40). All imaging acquisitions were focused on the operated left femur. All μCT images were performed at the test site using the Skyscan 1276 (Brucker).

Analysis with Microcomputed Tomography (μCT)

A 3D reconstruction of the femur was generated using 3D slicer software. A cylinder modeling the bone defect was first calculated. This cylinder was adjusted for each animal to be placed between the two internal screws. For each animal and at each time point, the cylinder was placed in the bone defect in the reconstructed 3D image of the femur to quantify the bone volume in voxels. This bone volume was then normalized to W0, which represented the baseline for each animal.

### Femur Harvesting

After μCT imaging, the operated left femur of each rat was explanted at the test site at W17. Briefly, the femur of the operated leg was dislocated from the femoral cavity of the pelvis and the knee joint. The explanted femur was then placed in 4% formaldehyde at room temperature for at least 48 hours.

### Treatment of the Femur for Histological Analysis

After fixation, samples were transferred to medium-action HCl for 24 h (3×8 h) at room temperature. Decalcification was checked near the femoral head for each sample to ensure complete decalcification. Then, the bone defect was isolated from the entire femur by sawing it along the innermost screws. The plate was then removed and the samples were included in paraffin at room temperature. Two longitudinal histological sections per femur (n=80 sections) were cut with a microtome. Two levels with an interval of 200 μM (with 2 sections per slide) were stained with toluidine blue. The sample sections included the entire width of the defect to allow assessment of bone healing.

### Microscopic Evaluation

Bone healing was evaluated under the microscope. Both levels were assessed by two trained experts and in blind. A histological scoring system adapted from Musson et al. (2019) is presented in Table 1. Each score was analyzed alone or in combination (global score then each evaluation criterion).

TABLE 1
Histological scoring system used to classify bone
healing (adapted from Musson et al. 2019).

| Category | Standard | Scores |
|---|---|---|
| Bone formation | 0% | 0 |
| (Percentage of defect | 1%-24% | 1 |
| bridged by bone) | 25%-49% | 2 |
| | 50%-74% | 3 |
| | 75%-100% | 4 |
| New bone type | No new bone | |
| (Nature of new bone | Predominantly woven | 1 |
| within defect) | Predominantly lamella | 2 |
| | remodelled | |
| Defect content | Mostly fibrous | 0 |
| | Mostly cartilaginous | 2 |
| | No content, fusion | 4 |

### Results

### 1. Body Weight

Based on body weight and post-surgery follow-up, animals were randomly assigned to five groups. Note that there were no significant differences between the groups in body weight after randomization (which ranged from 335.4 g to 356.5 g).

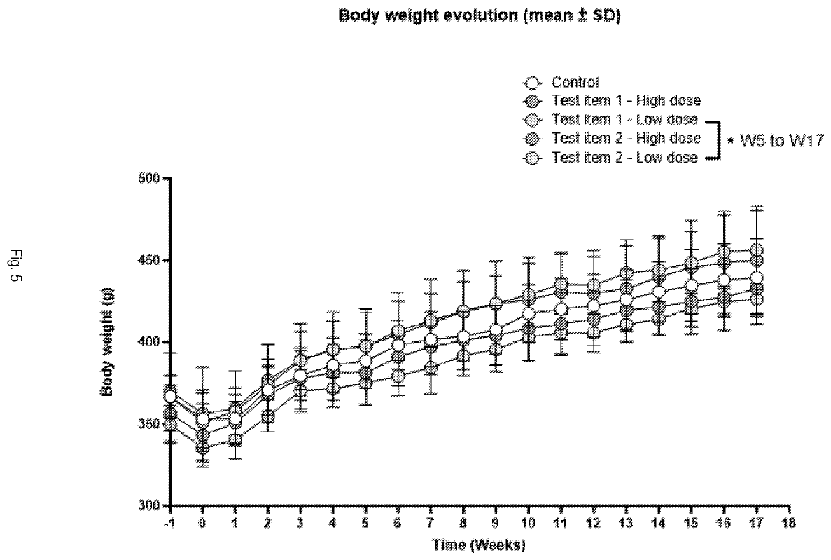

For all groups, body weight tended to decrease directly after the surgery at W0 (i.e., Day 3 after the surgery) (FIG. 5) (from 13.3 g to 15.4 g body weight loss). However, from W1 after the surgery and until the end of the study (W17), all rats gained weight (from 86.3 g to 105.3 g). From W5 to W17, a significant difference was shown between Group 3 (Test item 1 low dose) and Group 5 (Test item 2 low dose), with Group 3 showing an increase in body weight. Interestingly, Group 3 had already shown higher body weight at the time of randomization, although it was not significantly different from Group 5.

Therefore, it can be assumed that this difference is independent from the tests administered and may be related to the randomization of animals into the study groups. Furthermore, this difference was not recorded for the high-dose tests (Group 3 versus Group 5).

Importantly, all animals gained weight over the course of the study, which suggests that oral administration for 5 days a week for 16 weeks was very well tolerated by the rats.

### 1. μCT Analysis of the Bone Healing Process

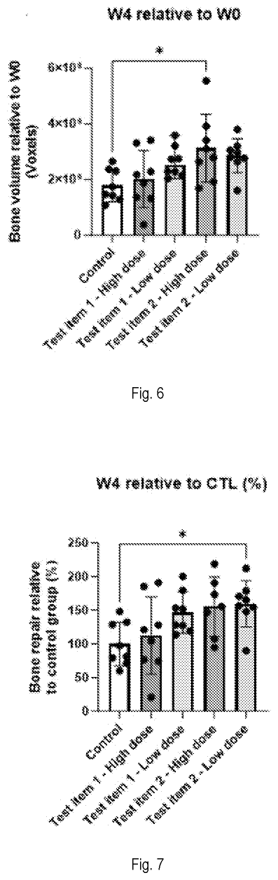

FIG. 6 presents the increase in bone volume relative to W0 at each timepoint (i.e., W4, W8, W12 and W17). These data are expressed in voxels, which represent the number of 3D pixels containing bone. At W4, group 4 (high-dose test item 2; 3127528±1221873 voxels) showed a significant increase in bone volume within the defect compared with the control group (group 1; 1788619±580204 voxels) (FIG. 6). A trend was observed between group 4 (high-dose test item 2; 3127528±1221873 voxels) and group 3 (low-dose test item 1; 2627017±549470 voxels) at W4 (p value=0.0864) (FIG. 6). No other significant differences in bone volume were observed until the end of the experiment.

FIG. 7 shows the percentage of bone repair compared with Group 1 (control group) at each time point (W4, W8, W12 and W17). At W4, Group 5 (low-dose test item 2; 159.7±34.46%) showed significantly greater bone volume than Group 1 (control group; 100.0±32.44%) (FIG. 7). Group 4 (high-dose test item 2; 155.5±44.03%) also tended to increase bone repair compared with group 1 (control group; 100.0±32.44%) at W4, although it did not reach statistical significance (p value=0.0893) (FIG. 7). No other significant differences were observed regardless of group and time (FIG. 7).

Overall, these data suggest that Test item 2 in both concentrations, administered orally immediately after bone fracture, improved bone healing at W4.

### CONCLUSION

This study demonstrated that administration of Test item 2 (lipid-based compound+Ultra D3) at both doses and administered orally for five days per week after CSBD (critical-sized bone defect) improved long bone healing after four weeks W4. This experiment confirms that the composition object of the present invention is a potent therapeutic agent to promote bone healing when administered early, preferably in the first four weeks, after fracture (during the inflammation/hematoma phase).

1. A composition comprising (I) and optionally (II), wherein:
(I) is a mixture comprising or, alternatively, consisting of:
(a) at least one cetylated fatty acid wherein said fatty acid is a saturated or unsaturated fatty acid having a number of carbons in the range from C6 to C22,
(b) a vitamin D and, optionally,
(c) at least one antioxidant in a weight percentage from 0.001% to 0.5% with respect to the total weight of the composition; and
(II) is at least one additive and/or excipient of pharmaceutical or food grade;
wherein said fatty acid of said (a) at least one cetylated fatty acid is selected from the group comprising or, alternatively, consisting of: myristic acid, oleic acid and a mixture thereof.

2. The composition according to claim 1, wherein said (b) vitamin D, is a vitamin D3, preferably is vitamin UltraD3®.

3. The composition according to any one of claims 1-2, wherein said (b) vitamin D is present in an amount from 0.0001% (0.001 mg/ml) to 0.01% (1 mg/ml).

4. The composition according to any one of claims 1-3, wherein said (c) at least one antioxidant is selected from the group comprising or, alternatively, consisting of: tert-butylhydroquinone (TBHQ), a mixture comprising tocopherol, lecithin, ascorbyl palmitate and citric acid (Aperoxid® TLA), a mixture of tocopherols and a natural rosemary extract; preferably said (c) is tert-butylhydroquinone (TBHQ).

5. The composition according to any one of claims 1-4, wherein said (c) at least one antioxidant is present in a weight percentage from 0.005% to 0.1% with respect to the total weight of the composition, preferably from 0.01% to 0.08%.

6. The composition according to any one of claims 1-5, wherein said (a) comprises or alternatively, consists of a mixture of cetylated myristic acid and cetylated oleic acid, wherein the molar ratio between said cetylated myristic acid and cetylated oleic acid is in the range from 4:1 to 1:1, preferably from 3:1 to 1.5:1, more preferably is (2.0±0.2):1; and
said (b) is vitamin D from UltraD3® and is present in an amount of about 0.0001% (0.001 mg/ml); and
said (c) is tert-butyl-hydroquinone (TBHQ) and is present in a weight percentage from 0.001% to 0.5% with respect to the total weight of the composition, preferably from 0.005% to 0.1%, more preferably from 0.01% to 0.08%.

7. The composition according to any one of claims 1-6, wherein said composition in addition to (a) and (b) and, optionally, (c) further comprises (d) a vegetable oil; preferably said vegetable oil is selected from the group comprising or, alternatively, consisting of olive oil, soybean oil, corn oil, linseed oil and sunflower oil having a high oleic acid content.

8. Composition according to any one of claims 1-7, wherein said composition comprises:
said (a) at least one cetylated fatty acid, preferably a mixture of cetylated myristic acid and cetylated oleic acid, in a weight % in the range from 50% to 90% with respect to the total weight of the composition, preferably from 60% to 85%, more preferably from 70% to 80%;
said (b) vitamin D, preferably vitamin UltraD3®, in an amount of 0.0001% (0.001 mg/ml);
said (c) antioxidant, preferably tert-butylhydroquinone (TBHQ), in a weight percentage from 0.001% to 0.5% with respect to the total weight of the composition, preferably from 0.005% to 0.1%, more preferably from 0.01% to 0.08%; and
said (d) vegetable oil, preferably olive oil, in a % by weight in the range from 9.5% to 50% with respect to the total weight of the composition, preferably from 14.5% to 40%, more preferably from 19.5% to 30%.

9. The composition according to any one of claims 1-8, wherein said composition is for use as a medicament.

10. The composition for use according to claim 9, wherein said composition is for use in a method of treatment of bone proliferation-regeneration; preferably it is for use in a method for promoting and improving bone proliferation-regeneration.

11. The composition for use according to claim 9, wherein said composition is for use in a method of treatment, preferably preventative, of osteoporosis.

12. The composition for use according to claim 9, wherein said composition is for use in a method of treatment, preferably curative, of a bone fracture.

13. The composition for use according to claim 9, wherein said composition is for use in a method of treatment in prosthetic surgery, bone growth, osteoarthritis and post-fracture consolidation.

14. The composition for use according to claim 9, wherein said composition is for use in a method of treatment of joint pain, musculoskeletal pain, preferably of an inflammatory nature.

15. The composition for use according to any one of claims 9-14, wherein said composition is formulated for oral use.
